(19) United States
(12) Patent Application Publication
(10) Pub. No.: US 2025/0228706 A1
(43) Pub. Date: Jul. 17, 2025

(54) SYSTEMS AND METHODS FOR DELIVERING AND DEPLOYING ADJUSTABLE SHUNTING SYSTEMS

(71) Applicant: Shifamed Holdings, LLC, Campbell, CA (US)

(72) Inventors: Katherine Sapozhnikov, Campbell, CA (US); Steven Pham, Campbell, CA (US); Tessa Bronez, Campbell, CA (US); Eric Schultz, Los Altos, CA (US); David Batten, San Francisco, CA (US)

(21) Appl. No.: 18/697,680

(22) PCT Filed: Oct. 13, 2022
(86) PCT No.: PCT/US2022/046596

§ 371 Date: Apr. 1, 2024

Related U.S. Application Data
(60) Provisional application No. 63/371,848, filed on Aug. 18, 2022.
(60) Provisional application No. 63/255,383, filed on Oct. 13, 2021.

(51) Int. Cl. A61F 9/007; A61B 17/00; A61B 17/34

(52) U.S. Cl. CPC A61F 9/00781; A61B 2017/00561; A61B 17/3468; A61B 2217/007

(57) ABSTRACT

The present technology is generally directed to shunt delivery systems and associated methods for delivering and deploying adjustable shunts. In some embodiments, the delivery systems include a device that can be easily held and manipulated by a physician or other user during an implant procedure. In some embodiments, the delivery system can be configured to facilitate priming of the adjustable shunt, such as before implantation of the adjustable shunt. In at least some embodiments, for example, the delivery system can include one or more priming ports or apertures fluidly coupled to the intraocular shunting system at least when the delivery system is in the first state. Fluid can be introduced into the delivery system via individual ones of the priming ports and flow toward and/or into the adjustable shunt to thereby “prime” the adjustable shunt.

### CROSS-REFERENCE TO RELATED APPLICATION(S)

The present application claims priority to U.S. Provisional Patent Application No. 63/255,383, filed Oct. 13, 2021, and U.S. Provisional Patent Application No. 63/371,848, filed Aug. 18, 2022, the disclosures of which are incorporated by reference herein in their entireties.

### TECHNICAL FIELD

The present technology generally relates to systems and methods for delivering and deploying implantable medical devices and, in particular, to delivery systems and associated methods for delivering and deploying intraocular shunts.

### BACKGROUND

Implantable shunting systems are widely used to treat a variety of patient conditions by shunting fluid from a first body region/cavity to a second body region/cavity. For example, shunting systems have been proposed for treating glaucoma. The flow of fluid through the shunting systems is primarily controlled by the pressure gradient across the shunt and the physical characteristics of the flow path defined through the shunt (e.g., the resistance of the shunt lumen). Conventional, early shunting systems (sometimes referred to as minimally invasive glaucoma surgery devices or “MIGS” devices) have shown clinical benefit; however, there is a need for improved shunting systems, systems for delivering such shunting systems, and techniques for addressing elevated intraocular pressure and risks associated with glaucoma. For example, there is a need for shunting systems capable of adjusting the therapy provided, including the flow rate between the two fluidly connected bodies. Further, there is a need for delivery systems for effectively and precisely delivering such shunting systems to target treatment locations within patients.

### BRIEF DESCRIPTION OF THE DRAWINGS

Many aspects of the present technology can be better understood with reference to the following drawings. The components in the drawings are not necessarily drawn to scale. Instead, emphasis is placed on illustrating clearly the principles of the present technology. Furthermore, components can be shown as transparent in certain views for clarity of illustration only and not to indicate that the component is necessarily transparent. Components may also be shown schematically.

FIGS. 1A-1C are perspective, side, and exploded views, respectively, of a delivery system configured for delivery of an adjustable shunting system in accordance with select embodiments of the present technology.

FIGS. 2A and 2B are side views illustrating operation of the delivery system of FIGS. 1A-1C.

FIGS. 3A-3C illustrate various stages of an operation for deploying a shunting system into a human eye using the delivery system of FIGS. 1A-1C in accordance with select embodiments of the present technology.

FIGS. 4A-4D are perspective, side, exploded, and enlarged views, respectively, of another delivery system configured for delivery of an adjustable shunting system in accordance with embodiments of the present technology.

FIGS. 5A and 5B are top views illustrating operation of the delivery system of FIGS. 4A-4D in accordance with embodiments of the present technology.

FIG. 6 is a top view illustrating a portion of a priming procedure including the delivery system of FIGS. 3A-3C in accordance with embodiments of the present technology.

FIG. 7 is a perspective view of an adjustable shunting system that can be delivered using the delivery system of FIGS. 1A-2C and/or FIGS. 4A-6 and configured in accordance with select embodiments of the present technology.

FIGS. 8A and 8B are perspective views of a priming assembly positioned within the adjustable shunting system of FIG. 7 and configured in accordance with embodiments of the present technology.

FIG. 8C is a partially-schematic cross-sectional view of the adjustable shunting system of FIG. 7 and the priming assembly of FIGS. 8A and 8B taken along line 8C-8C in FIG. 8A.

FIG. 8D is a partially-schematic cross-sectional view of the adjustable shunting system of FIG. 7 and the priming assembly of FIGS. 8A and 8B taken along line 8D-8D in FIG. 8B.

FIG. 9 is a perspective view of another priming assembly configured in accordance with embodiments of the present technology.

### DETAILED DESCRIPTION

The present technology is directed to delivery systems and associated methods for delivering and deploying adjustable shunting systems. In some embodiments, the delivery systems include a device that can be easily held and manipulated by a physician or other user during an implant procedure. The delivery systems can include a body, a drive assembly, and a driven assembly. The driven assembly can be operably coupled to the drive assembly, such that movement of the drive assembly can cause corresponding movement of the driven assembly. Additionally, the driven assembly can be operably coupled to an adjustable shunting system configured for implantation within a patient, such that movement of the driven assembly can cause corresponding movement of the adjustable shunting system. In at least some embodiments, for example, the delivery system can be configured to transition between (i) a first state or configuration in which the adjustable shunting system is positioned within the body of the delivery system, and (ii) a second state or configuration in which the adjustable shunting system is positioned outside of and/or extends at least partially beyond the body of the delivery system. A user can rotate the drive assembly to cause linear movement of the driven assembly and cause the delivery system to transition from the first state to and/or toward the second state. With the delivery system in the second state, the delivery system can be used to implant the adjustable shunting system in the patient during the implantation procedure, such as within an eye of the patient.

In some embodiments, the delivery system can be configured to facilitate priming of the adjustable shunting system, e.g., before the implantation of the shunting system within a patient. In at least some embodiments, for example, the delivery system can include one or more priming ports or apertures fluidly coupled to the adjustable shunting system at least when the delivery system is in the first state. Fluid (e.g., priming fluid) can be introduced into the delivery system via individual ones of the priming ports and flow toward and/or into the adjustable shunting system, to thereby “prime” the shunting system. Priming the adjustable shunting system (e.g., before implantation), is expected to reduce the resistance to initiating fluid flow through the adjustable shunting system (e.g., during and/or after implantation). In these and other embodiments, a priming assembly can be positioned at least partially within the adjustable shunting system. In at least some embodiments, for example, the priming assembly can be configured to couple the adjustable shunting system to the driven assembly. At least a portion of the priming assembly can be moved relative to (e.g., at least partially removed from) the adjustable shunting system to prime the shunting system. For example, moving the priming assembly relative to the adjustable shunting system can cause fluid to be drawn into and/or through at least a portion of the shunting system, thereby initiating fluid flow through the shunting system and/or reducing the resistance to initiating fluid flow through the shunting system.

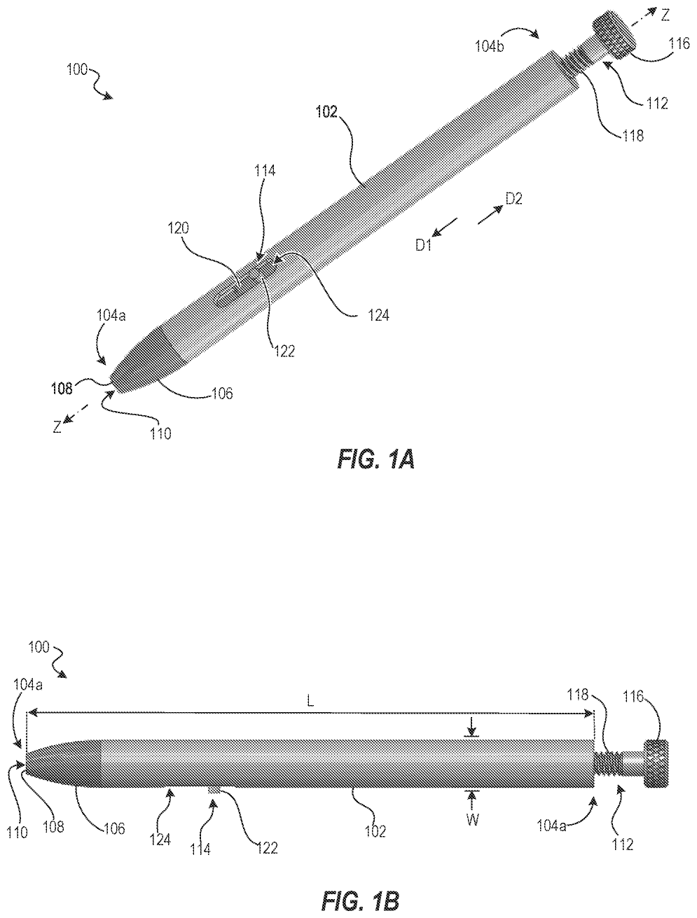

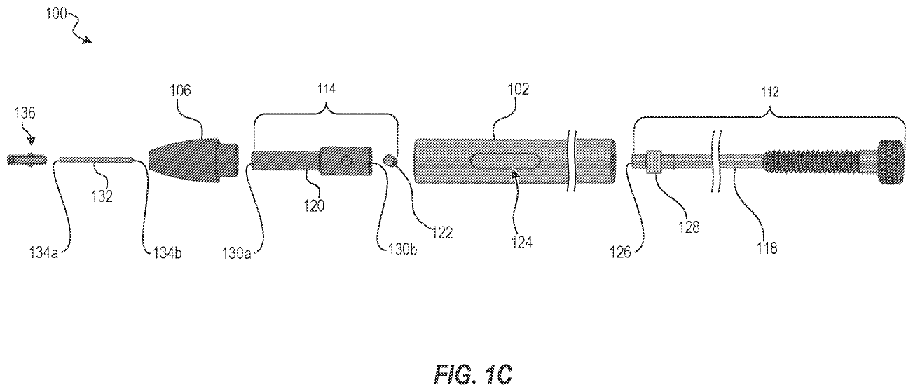

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims but are not described in detail with respect to FIGS. 1A-9.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

As used herein, the use of relative terminology, such as “about”, “approximately”, “substantially” and the like refer to the stated value plus or minus ten percent. For example, the use of the term “about 100” refers to a range of from 90 to 110, inclusive. In instances in which the context requires otherwise and/or relative terminology is used in reference to something that does not include a numerical value, the terms are given their ordinary meaning to one skilled in the art.

Reference throughout this specification to the term “resistance” refers to fluid resistance unless the context clearly dictates otherwise. The terms “drainage rate” and “flow rate” are used interchangeably to describe the movement of fluid through a structure at a particular volumetric rate. The term “flow” is used herein to refer to the motion of fluid, in general.

Although certain embodiments herein are described in terms of shunting fluid from an anterior chamber of an eye, one of skill in the art will appreciate that the present technology can be readily adapted to shunt fluid from and/or between other portions of the eye (including the posterior chamber), or, more generally, from and/or between a first body region and a second body region. Moreover, while the certain embodiments herein are described in the context of glaucoma treatment, any of the embodiments herein, including those referred to as “glaucoma shunts” or “glaucoma devices” may nevertheless be used and/or modified to treat other diseases or conditions, including other diseases or conditions of the eye or other body regions. For example, the systems described herein can be used to treat diseases characterized by increased pressure and/or fluid build-up, including but not limited to heart failure (e.g., heart failure with preserved ejection fraction, heart failure with reduced ejection fraction, etc.), pulmonary failure, renal failure, hydrocephalus, and the like. Moreover, while generally described in terms of shunting aqueous, the systems described herein may be applied equally to shunting other fluid, such as blood or cerebrospinal fluid, between the first body region and the second body region.

FIGS. 1A-1C are perspective, side, and exploded views, respectively, of a delivery system 100 (“system 100”) configured for delivery of an adjustable shunting system in accordance with embodiments of the present technology. Referring to FIGS. 1A and 1B together, the system 100 includes a delivery system body or housing 102, a first (e.g., distal) end portion 104a, and a second (e.g., proximal) end portion 104b opposite the first end portion 104a. The housing 102 can have a length L of, for example, at least 2 inches, 3 inches, 4 inches, 5 inches, 5.5 inches, 6 inches, 7 inches, any length therebetween, or another suitable length. The housing 102 can have a width W of at least, for example, 0.1 inch, 0.2 inch, 0.3 inch, 0.35 inch, 0.375 inch, 0.4 inch, 0.5 inch, 1 inch, any width therebetween, or another suitable width. The system 100 can further include a nosecone or tip 106 coupled to the housing 102 and/or a portion of the housing 102 at or near the first end portion 104a. The tip 106 can define a distal terminus 108 of the system 100. Additionally, the housing 102 and/or the tip 106 can be hollow and define a lumen 110 extending through the housing 102, e.g., between the first and second end portions 104a, 104b.

The system 100 can further include a drive assembly 112 and a driven assembly 114. The drive assembly 112 can include, for example, a drive head 116 and a drive shaft 118 coupled to and/or extending from the drive head 116. In the illustrated embodiment, at least a portion of the drive shaft 118 is threaded and configured to threadably engage the housing 102 within the lumen 110 (e.g., via the second end portion 104b). With continued reference to the illustrated embodiment, the drive head 116 can be rotated relative to the housing 102 to cause the drive shaft 118 to move along a longitudinal axis Z of the system 100, e.g., in a first direction D1 toward the distal terminus 108 and/or in a second direction D2 away from the distal terminus 108. In other embodiments, the drive assembly 112 can have another suitable configuration. For example, the drive shaft 118 can include one or more projections or recesses configured to be slidably received by correspondingly-shaped structures within the housing 102.

The driven assembly 114 can include a pusher or driven component 120. The driven component 120 can be operably coupled to the drive shaft 118 such that movement of the drive shaft 118 causes corresponding movement of the driven component 120, e.g., in the first direction D1 and/or the second direction D2. For example, rotational movement of the drive shaft 118 that causes the drive shaft 118 to move toward the distal terminus 108 can also cause the driven component 120 to move toward the distal terminus 108. In the illustrated embodiment, the driven assembly 114 further includes a key or pin 122 configured to at least partially prevent rotational movement of the driven component 120. The pin 122 can be slidably received by a slot 124 through the housing 102 to thereby inhibit or prevent rotation of the driven component 120 relative to the housing 102. In the illustrated embodiment, the slot 124 can be formed fully-through the housing 102, such that the pin 122 can extend through the slot 124 and protrude radially outward from the housing 102. In other embodiments, the slot 124 can be formed partially though the housing 102, such that the pin 122 can be positioned within the housing 102. In further embodiments, the pin 122 and/or the slot 124 can be omitted.

Referring to FIG. 1C, the drive shaft 118 of the drive assembly 112 can include a distal terminus 126 configured to contact or otherwise engage at least a portion of the driven assembly 114. In some embodiments, the drive shaft 118 includes a sealing element 128, such as an O-ring, configured to sealing engage with the interior of the housing 102. The sealing element 128 can be positioned at or near (e.g., proximally from) the distal terminus 126. The driven component 120 can include a first (e.g., distal) end 130a and a second (e.g., proximal) end 130b opposite the first end 130a. When the system 100 is assembled (as shown in FIGS. 1A and 1B), the second end 130b can be contacted/engaged by a distal terminus 126 of the drive assembly 112. In at least some embodiments, the distal terminus 126 can be positioned within the second end 130b.

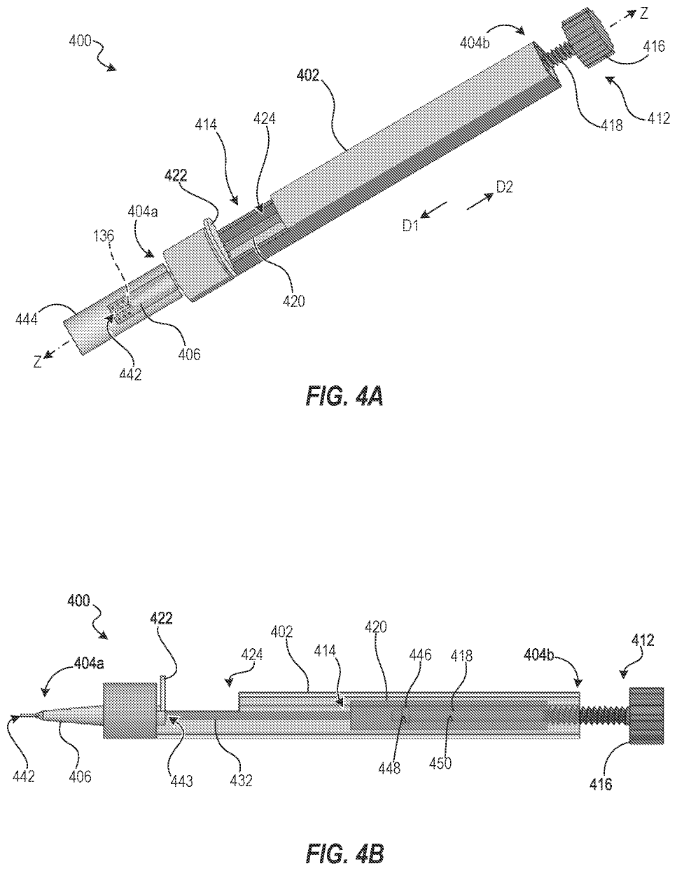

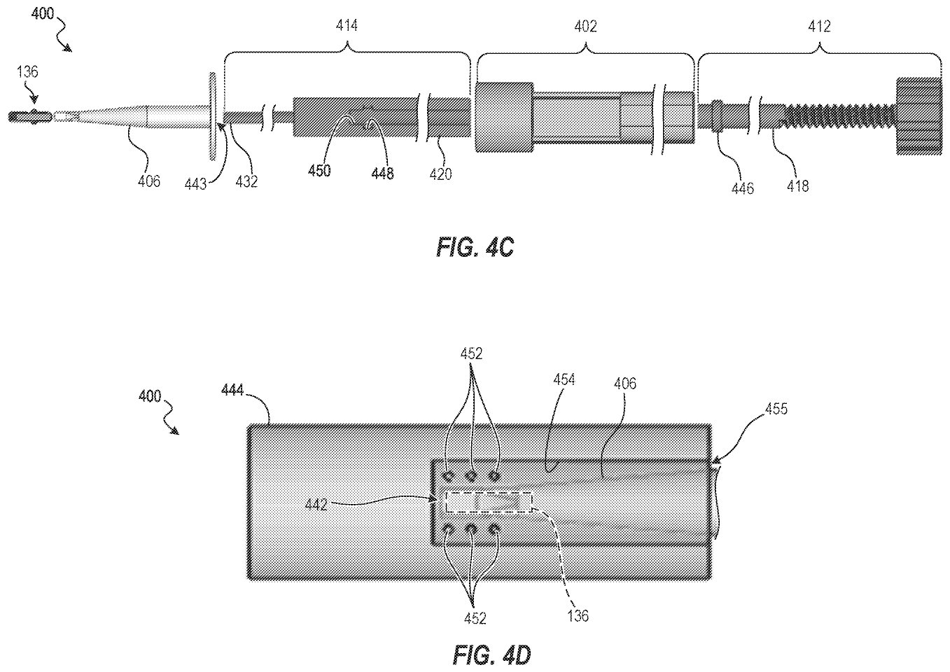

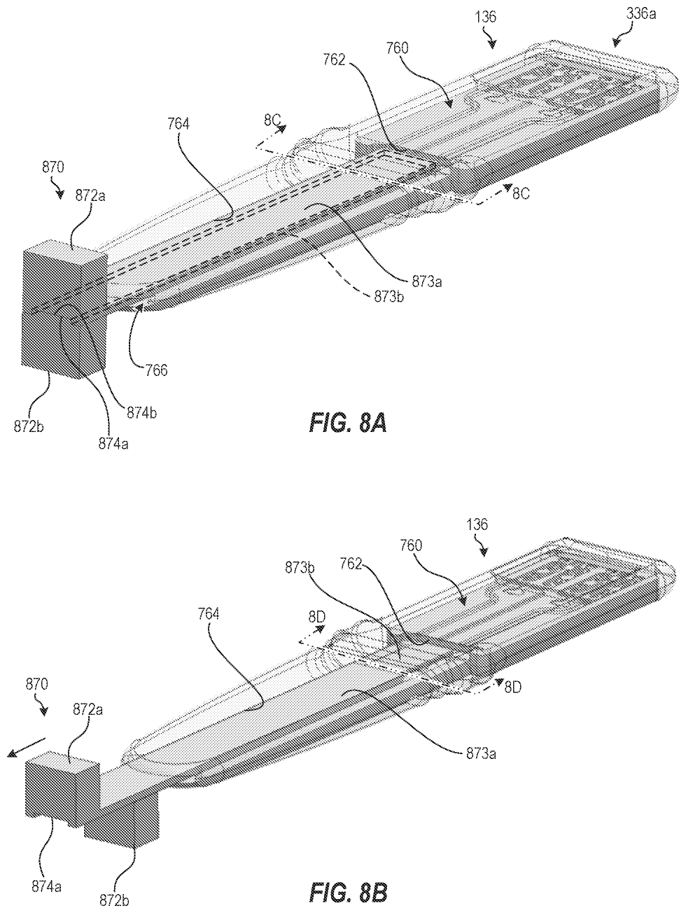

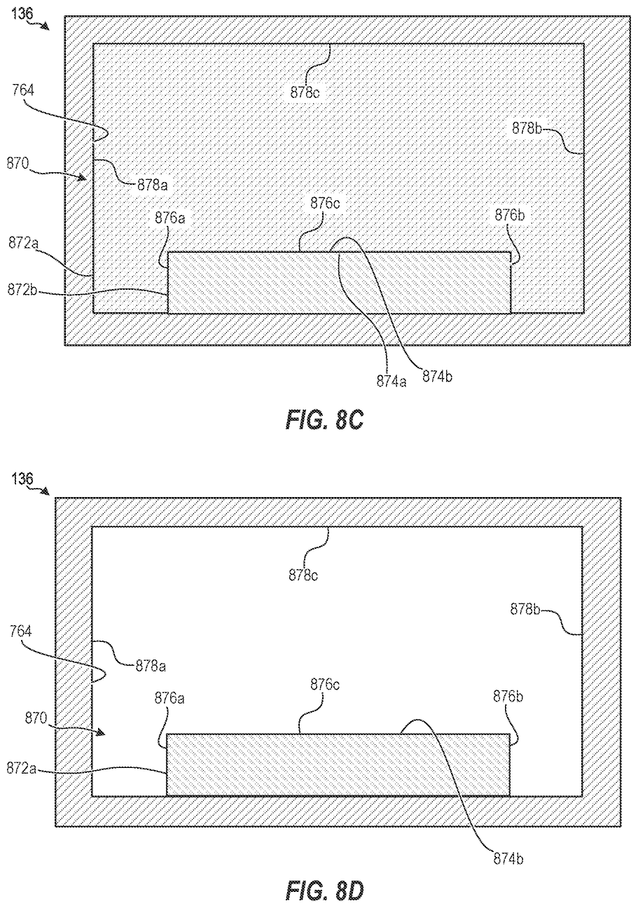

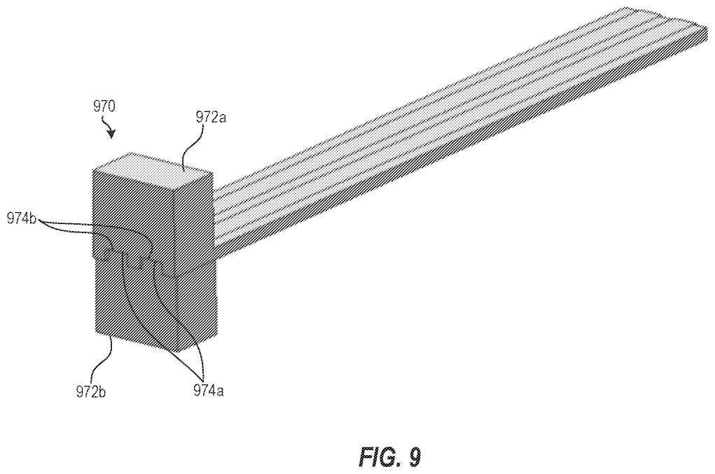

The system 100 can further include a push rod or shunt coupling member 132 having a first or shunt-engaging end portion 134a (“first end portion 134a”) and a second or driven component-engaging end portion 134b (“second end portion 134b”) opposite the first end portion 134a. The second end portion 134b can be configured to be coupled to and/or otherwise engage the first end 130a of the driven component 120, such that movement of the driven component 120 can cause corresponding movement of the shunt coupling member 132. The first end portion 134a of the shunt coupling member 132 can be configured to be releasably engaged with an adjustable shunting system 136 (“shunt 136”), such that movement of the shunt coupling member 132 results in corresponding movement of the shunt 136. In at least some embodiments, for example, the first end portion 134a of the shunt coupling member 132 can be positioned within the shunt 136, such that the shunt coupling member 132 can be withdrawn from the shunt 136 to uncouple the shunt 136 from the system 100. This is described in further detail below with reference to FIGS. 8A-9. In other embodiments, the shunt coupling member 132 can be configured to be releasably coupled to an external surface of the shunt 136, such as by forming a substantially fluid-impermeable seal around at least a portion of the shunt 136. In these and other embodiments, the shunt coupling member 132 can be configured to facilitate priming of the shunt 136, e.g., before implantation. Priming the shunt 136 is described in further detail below (e.g., with reference to FIGS. 4D, 6, and 8A-9).

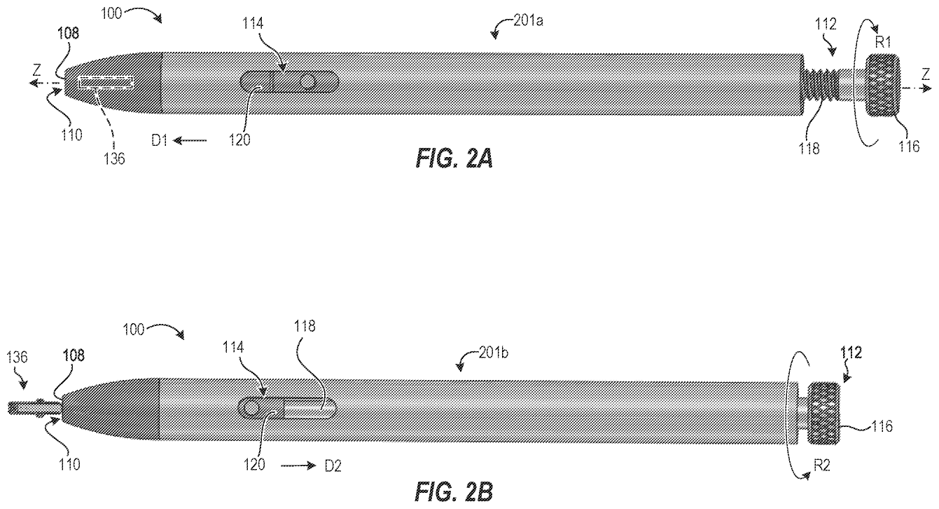

FIGS. 2A and 2B are side views illustrating operation of the system 100 of FIGS. 1A-1C. More specifically, referring to FIGS. 2A and 2B together, the drive assembly 112 can be actuated (e.g., by a user) to cause movement of the driven assembly 114 and transition the system 100 between a first state 201a (FIG. 2A) and a second state 201b (FIG. 2B). In the illustrated embodiment, for example, a user can rotate the drive head 116 in a first rotary direction R1 to cause the drive shaft 118 to move in the first direction D1, as shown in FIG. 2A. This movement of the drive shaft 118 causes corresponding movement of the driven component 120 (e.g., in the first direction D1) and thereby transitions the system 100 from the first state 201a toward and/or to the second state 201b, shown in FIG. 2B. In the second state 201b, the shunt 136 can be positioned for insertion/implantation within a patient (not shown). For example, in the second state 201b, at least part or all of the shunt 136 can be positioned outside of the lumen 110 and/or extend beyond (e.g., distally beyond) the distal terminus 108 of the system 100.

As shown in FIG. 2B, the user can transition the system 100 from the second state 201b toward and/or to the first state 201a by rotating the drive head 116 in a second rotary direction R2 opposite the first rotary direction R1 to cause the drive shaft 118 to move in the second direction D2. This movement of the drive shaft 118 can cause corresponding movement of the driven component 120 (e.g., in the second direction D2) and thereby transition the system 100 from the second state 201b toward and/or to the first state 201a (FIG. 2A). In the first state 201a, the shunt 136 (shown schematically) can be positioned within (e.g., fully within) the housing 102, such that the housing 102 can protect or shield the shunt 136 from outside interference (e.g., damage, contaminants, particulate matter, and/or the like), such as before and/or during the implantation procedure, transportation, shipping, etc.

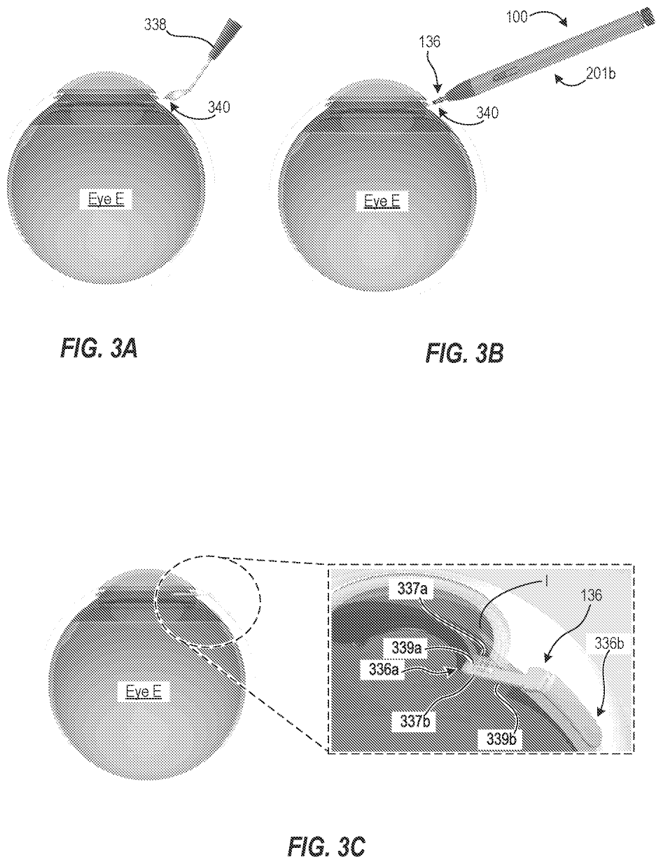

FIGS. 3A-3C illustrate various stages of an operation for deploying an adjustable shunting system into a patient's eye E using the system 100 in accordance with embodiments of the present technology. In a particular example, the system 100 can be used to deploy the shunt 136 such that, after implantation, the shunt 136 is positioned to route fluid from an anterior chamber of the patient's eye E to a suitable outflow location, such as a subconjunctival bleb space (e.g., to treat glaucoma).

Referring first to FIG. 3A, one or more tools 338 (e.g., a keratome) can be used to make one or more incisions 340 in the eye E. Referring next to FIG. 3B, the system 100 can be transitioned toward and/or to the second state 201b (as described above with reference to FIG. 2B) and used to insert the shunt 136 into the eye E through the incision 340. For example, as shown in FIG. 3C, the shunt 136 can be positioned such that a first or inflow portion 336a of the shunt 136 is positioned in a first body region and a second or outflow portion 336b of the shunt 136 is positioned in a second body region. In this way, after implantation, fluid within the first body region can flow/drain through the shunt 136 toward and/or into the second body region. In some embodiments, the shunt 136 can be configured to assume a curved, bent, or preformed shape during/after implantation in the patient, as shown in FIG. 3C. Additionally, or alternatively, the system 100 can be configured to adjust the shape of the system 100 in situ, and/or one or more of the tools 338 can be used to adjust the shape of the system 100 in situ.

Although in FIG. 3C the first portion 336a of the shunt 136 is illustrated as being positioned anterior to (e.g., in front of/above) the patient's iris I, in other embodiments the first portion 336a can be positioned posterior to (e.g., behind/below) the iris I (also referred to as “sub-iris” positioning). The sub-iris positioning of the shunt 136 is expected to reduce or prevent corneal endothelial disease and/or failure, for example, by reducing or preventing disruption to nutrient and/or other fluid and/or chemical transport to and/or through the corneal endothelium. In the illustrated embodiment, the shunt 136 is configured to receive fluid (e.g., aqueous) through one or more openings or inlets in an upper surface 339a of the shunt 136. In these and other embodiments, including when the shunt 136 has a sub-iris position, the shunt 136 can be configured to receive fluid through one or more lateral openings, e.g., positioned in one or more sides 337a, 337b of the shunt 136, and/or through one or more openings in a bottom surface 339b of the shunt 136.

In general, the shunt 136 can be configured to actuate and/or change its resistance to fluid flow in response to energy (e.g., laser energy) delivered from a source external to the patient. An example of such a configuration is described in detail in U.S. Patent App. Publication No. US 2021/0251806, filed Feb. 12, 2021, and incorporated herein by reference for all purposes. In embodiments in which the shunt 136 is positioned sub-iris, the shunt 136 can be actuated using a number of techniques. For example, a portion of the iris I can be removed (e.g., iridectomy) to provide line-of-sight access to the shunt 136. Additionally, or alternatively, the shunt 136 can be positioned such that at least a portion of the shunt 136 (e.g., at least part of the first portion 336a) can be exposed when the eye E undergoes pupil dilation. In such embodiments, the pupil of the eye E can be dilated and then energy can be delivered to actuate the shunt 136. In these and other embodiments, the energy can be targeted using a first energy source (e.g., a first laser) configured to transmit first energy (e.g., targeting energy) at or near a first wavelength to which the iris I is translucent or transparent, and then a same or different energy source can transmit second energy (e.g., actuating energy) at a second wavelength different than the first wavelength.

In some embodiments, the shunt 136 can be configured to reduce or prevent cellular growth onto, over, and/or around at least a portion of the shunt 136. In at least some embodiments, for example, all or a portion of the shunt 136 can include one or more radioisotopes configured to inhibit or prevent cellular growth. The radioisotopes can include Phosporous-32, Strontium-89, Strontium-90, Yttrium-90, and/or another suitable radioisotope. Individual ones of the radioisotopes can emit alpha, beta, and/or gamma radiation, each of which are expected to inhibit or prevent cellular growth on, over, and/or near the shunt 136. For a given patient, the radioisotope(s) used with the shunt 136 can be selected based at least partially on the half-life of the radioisotope, the type of radiation, and/or the energy of the emitted alpha, beta, and/or gamma particles. The radioisotopes can be naturally-occurring or manufactured (e.g., using a cyclotron, reactor-produced, etc.).

FIG. 4A is a perspective view of another delivery system 400 (“system 400”) configured in accordance with embodiments of the present technology. FIG. 4B is a side view of the system 400 with select aspects of the system 400 illustrated as transparent merely for purposes of illustration. At least some aspects of the system 400 can be generally similar or identical in structure and/or function to the system 100 of FIGS. 1A-3C. For example, the system 400 can be configured to carry the shunt 136 (shown schematically in FIG. 4A) and/or used to implant the shunt 136 within a patient's eye, as described previously herein (e.g., with reference to FIGS. 3A-3C). Accordingly, like names and/or reference numbers (e.g., the housing 102 versus housing 402) are used to indicate generally similar or identical aspects.

Referring to FIGS. 4A and 4B together, the system 400 includes a housing 402 having a first (e.g., distal) end portion 404a, a second (e.g., proximal) end portion 404b opposite the first end portion 404a, and a nosecone or tip assembly 406 (“tip 406”) at or near the first end portion 404a. The tip 406 can include a slot or opening 442 through which an adjustable intraocular shunting system (e.g., the shunt 136) can be deployed. In the illustrated embodiment, the tip 406 is a separate component configured to be positionable within the housing 402 via a slot or opening 424 formed therethrough, such that the tip 406 can extend through and beyond the distal end portion 404a of the housing 402. The tip 406 can include a key or flange 422 configured to extend through the slot 424 and thereby at least partially prevent rotational movement of the tip 406 relative to the housing 402. In other embodiments, the tip 406 and the housing 402 can together comprise a unitary, single-piece assembly or component.

The system 400 can further include a drive assembly 412 and a driven assembly 414. The drive assembly 412 can include a drive head 416 and a drive shaft 418. The drive shaft 418 can be threaded and configured to threadably engage the housing 402. During operation, the drive head 416 can be rotated to cause the drive shaft 418 to move along a longitudinal axis Z of the system 100, e.g., in the first direction D1 and/or in the second direction D2. The driven assembly 414 can include a pusher or driven component 420. The driven component 420 can be operably coupled to the drive shaft 418 such that movement of the drive shaft 418 causes corresponding movement of the driven component 420, e.g., in the first direction D1 and/or the second direction D2.

FIG. 4C is an exploded top view of the system 400. Referring to FIGS. 4B and 4C together, the drive shaft 418 can include a key or protrusion 446 (e.g., an annular or radial key/protrusion) configured to be received within a slot or recess 448 (e.g., an annular or radial slot/recess) of a coupling channel 450 of the driven component 420. The protrusion 446 and/or the recess 448 can be configured to allow the drive assembly 412 to rotate (e.g., freely rotate) relative to the driven assembly 414 without or substantially without (i) causing corresponding rotational movement of the driven component 420 and/or (ii) exerting torque/torsion forces on the driven component 420. Accordingly, rotational movement of the drive assembly 412 (e.g., relative to the housing 402) can cause linear or substantially linear movement of the driven assembly 414 (e.g., along the longitudinal axis of the housing 402), as described previously herein (e.g., with reference to FIGS. 1A-2B). The linear movement of the driven assembly 414 can cause a push rod 432 to move the shunt 136 (FIG. 4C) through the tip 406, e.g., to deploy the shunt 136 from within the system 400. The push rod 432 can extend through a second or proximal opening 443 in the tip 406 to contact the shunt 136.

Referring again to FIG. 4A, the system 400 can optionally include a cap or covering component 444 configured to be releasably couplable to the system 400 and/or positioned around at least a portion of the tip 406. The cap 444 can at least partially protect the tip 406 and/or the shunt 136 from damage, e.g., during shipping and/or movement of the system 400. The cap 444 may be removed, as shown in FIG. 4B, as part of a procedure involving the system 400 and/or prior to deploying a shunt through the tip 406.

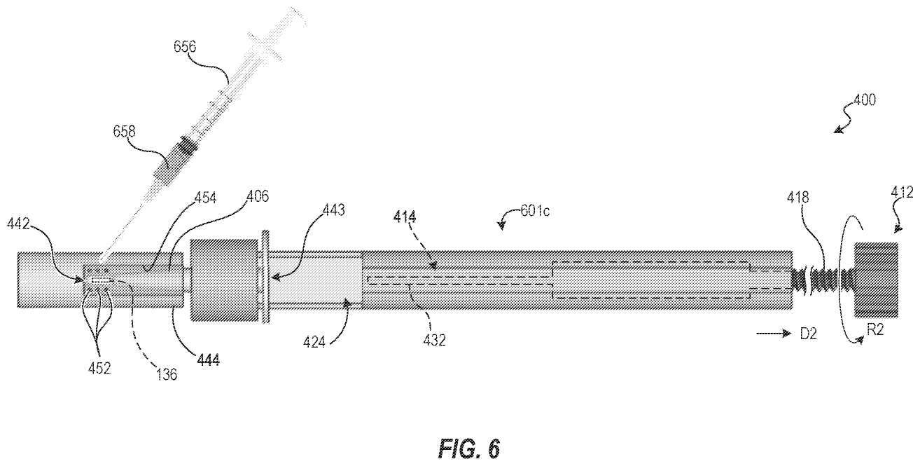

FIG. 4D is an enlarged view of the cap 444 and the tip 406, with other aspects of the system omitted for the purpose of clarity. As shown in FIG. 4D, the cap 444 can include one or more ports 452 (which can also be referred to as “priming ports”, “priming inlets”, and/or the like). Individual ones of the ports 452 can be fluidly coupled to an interior of the tip 406, e.g., via the opening 442. In at least some embodiments, for example, the cap 444 can define a chamber 454 configured to receive at least a portion of the tip 406. Fluid (e.g., priming fluid) introduced through one or more of the ports 452 can enter the chamber 454 and flow into the tip 406 via the opening 442. The fluid can be drawn through one or more of the ports 452, such as in response to a reduced pressure or vacuum generated within the chamber 454, injected through one or more of the ports 452, such as using a syringe or other fluid delivery tool, and/or another suitable fluid delivery technique. As described in further detail below with reference to FIG. 6, the fluid introduced via the ports 452 can be used to prime a shunt (e.g., shunt 136) carried by the system 400. The cap 444 can be configured to form a substantially fluid-impermeable seal 455 with at least a portion of the tip 406, such that all or substantially all fluid flow into and/or out of the chamber 454 is through one or more of the ports 452 and/or the tip 406.

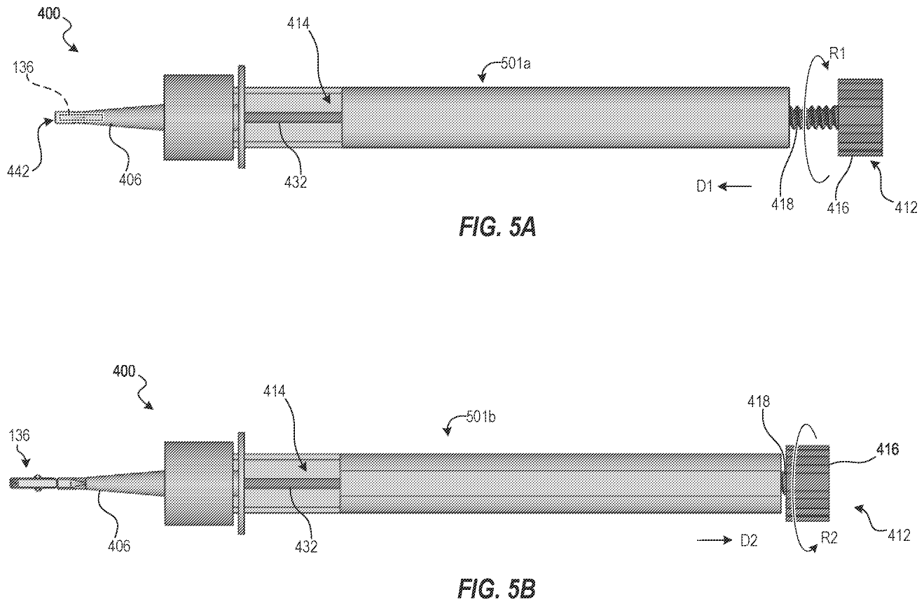

FIGS. 5A and 5B are side views illustrating operation of the system 400 of FIGS. 4A-4D. Referring to FIGS. 5A and 5B together, the drive assembly 412 can be actuated (e.g., by a user) to cause movement of the driven assembly 414 and transition the system 400 between a first state 501a (FIG. 5A) and a second state 501b (FIG. 5B). In the illustrated embodiment, for example, when a user rotates the drive head 416 in a first rotary direction R1, the drive shaft 418 moves in the first direction D1 (as shown in FIG. 5A). As noted previously, this movement of the drive shaft 418 results in corresponding movement of the driven component 420 and/or the push rod 432 (e.g., in the first direction D1), thereby transitioning the system 400 from the first state 501a toward and/or to the second state 501b (as shown in FIG. 5B). In the second state 501b, the shunt 136 is positioned for insertion/implantation within a patient. For example, in the second state 501b, at least part or all of the shunt 136 can be positioned outside of the tip 406, e.g., outside of and/or distally beyond the opening 442 (FIG. 5A).

The system 400 can be transitioned from the second state 501b toward and/or to the first state 501a by rotating the drive head 416 in a second rotary direction R2 opposite the first rotary direction R1 to cause the drive shaft 418 to move in the second direction D2, as shown in FIG. 2B. This movement of the drive shaft 418 results in corresponding movement of the driven component 420 (e.g., in the second direction D2), thereby transitioning the system 400 from the second state 501b toward and/or to the first state 501a, shown in FIG. 5A. As noted previously, in the first state 501a, the shunt 136 is positioned within (e.g., fully within) the tip 406 and accordingly protected/shielded from outside interference (e.g., damage, contaminants, particulate matter, etc.).

FIG. 6 is a top view of the system 400 during a priming procedure in accordance with embodiments of the present technology. In some embodiments, the shunt 136 is primed before implantation. Priming the shunt 136 can include introducing fluid (e.g., priming fluid) into at least a portion of the shunt 136 to thereby reduce resistance to initiating fluid flow through the shunt 136, e.g., after the shunt 136 has been implanted. In the illustrated embodiment, for example, a fluid delivery tool 656, such as a syringe, is loaded with priming fluid 658 and used to flow/inject the priming fluid 658 into the chamber 454 of the cap 444 via one or more of the ports 452 (three labeled in FIG. 6). Once within the chamber 454, the priming fluid 658 can flow into the shunt 136 via the opening 442 in the tip 406. For example, the force/pressure generated by the fluid delivery tool 656 and with which the priming fluid 658 is injected into the chamber 454 can be greater than a fluid inflow resistance of the shunt 136, such that all or part of the priming fluid 658 is expected to flow into the shunt 136. Additionally, or alternatively, injecting fluid into the chamber 454 can create a pressure gradient between the chamber 454 and the interior of the shunt 136; the pressure gradient can exceed the fluid inflow resistance of the shunt 136 and cause all or part of the priming fluid 658 to flow into the shunt 136.

In these and other embodiments, the priming fluid 658 can be introduced through the second opening 443 of the tip 406. For example, the drive assembly 412 can be rotated in the second rotary direction R2 to transition the system 400 to a third or priming state 601c in which the push rod 432 (shown schematically) is not positioned within and/or spaced apart from the second opening 443, and/or the driven assembly 414 (shown schematically) is not positioned within and/or is positioned proximally from the slot 424, as shown in FIG. 6. Retracting the push rod 432 from the second opening 443 of the tip 406 can allow the priming fluid 658 to be introduced into the tip 406 via the second opening 443. In some embodiments, priming the shunt 136 can include introducing the priming fluid 658 via the second opening 443 until at least a portion of the priming fluid 658 flows through the shunt 136, into the chamber 454, and/or out of the cap 444 through one or more of the ports 452. Additionally, or alternatively, priming the shunt 136 can include introducing the priming fluid 658 through individual ones of the ports 452 until at least a portion of the priming fluid 658 flows into the tip 406 via the opening 442, through shunt 136, toward the second opening 443, and/or out of tip 406 via the second opening 443.

In some embodiments, a reduced pressure or vacuum can be generated within the chamber 454 and used to draw priming fluid 658 into the chamber 454 from the fluid delivery tool 656 or another fluid source. Additionally, or alternatively, a reduced pressure or vacuum can be generated within the shunt 136 and used to drawing priming fluid 658 into at least a portion of the shunt 136. The chamber 454 and/or the shunt 136 can be evacuated via the second opening 443 and/or one or more of the ports 452. In some embodiments, the vacuum generated within the chamber 454 and/or the shunt 136 can be used to directly draw the priming fluid 658 into the chamber 454 and/or the shunt 136 (e.g., during and/or in concert with the evacuation of the chamber 454 and/or the shunt 136). In other embodiments, the chamber 454 and/or the shunt 136 can be sealed to store the vacuum such that, at some time after the vacuum is generated, the vacuum can be used to draw the priming fluid 658 into the chamber 454 and/or the shunt 136. In these and other embodiments, the vacuum generated within the chamber 454 and/or the shunt 136 can be used to draw fluid into the chamber 454 and/or the shunt 136 via one or more of the ports 452 and/or the second opening 443.

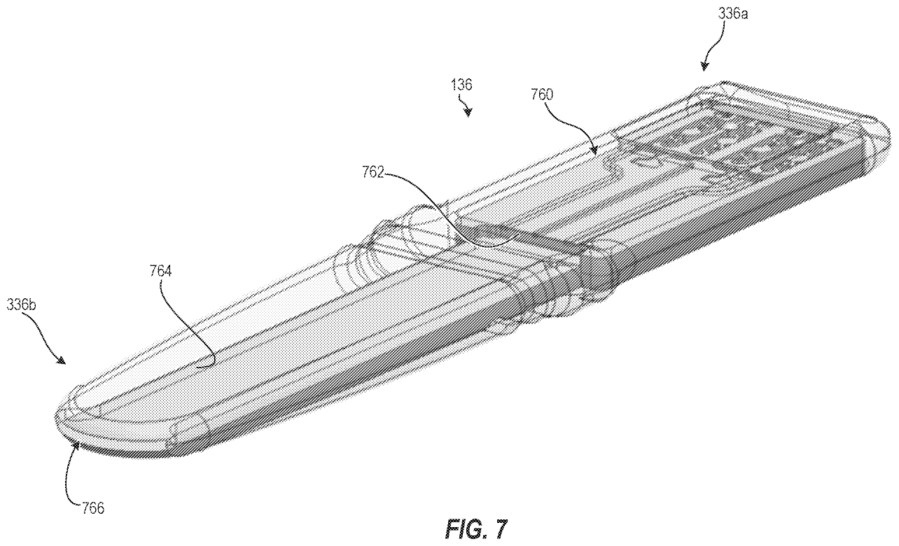

FIG. 7 is a perspective view of the shunt 136. The shunt 136 can include a plate or flow control assembly 760 configured to provide adjustable resistance to fluid (e.g., aqueous) flow through the shunt 136. The flow control assembly 760 can include an interface portion 762 that fluidly couples the flow control assembly 760 to an outflow channel 764 and a fluid outlet 766 of the shunt 136. In some embodiments, the shunt 136 can be configured to receive fluid via the first end portion 336a, such that fluid can flow through the shunt 136 from the flow control assembly 760 toward and/or to the outflow channel 764 and/or out of the shunt 136 through the second portion 336b. Additionally, or alternatively, the shunt 136 can be configured to receive fluid via the second portion 336b, such that fluid can flow through the shunt 136 from the outflow channel 764 toward and/or to flow control assembly 760 and/or out of the shunt 136 through the first portion 336a. In some embodiments, the direction of fluid flow through the shunt can be based at least partially on the relative positions of the first and second portions 336a, 336b, as described previously herein (e.g., with reference to FIG. 3C). Generally, at least some features of the shunt 136 can be generally similar or identical in structure and/or function to one or more features of the adjusting shunting systems described in U.S. Patent App. Publication No. US 2021/0251806, the entirety of which was previously incorporated by reference herein.

FIGS. 8A and 8B are perspective views of the shunt 136 and a priming interface or assembly 870 (“priming assembly 870”) configured in accordance with embodiments of the present technology. In some embodiments, the priming assembly 870 can be at least generally similar in structure and/or function to, identical in structure and/or function to, and/or otherwise incorporated as part of the shunt coupling member 132 (FIG. 1C) and/or the push rod 432 (FIGS. 4B and 4C), although in other embodiments the priming assembly 870 can be a standalone component or otherwise secured to a portion of the delivery system 100, 400.

Referring to FIGS. 8A and 8B together, the priming assembly 870 can include a first priming element 872a and a second priming element 872b, each of which can be configured to be positioned within the outflow channel 764 of the shunt 136. The first and second priming elements 872a, 872b can be configured to form a substantially fluid-impermeable seal when positioned within the outflow channel 764. In the illustrated embodiment, for example, one or both of the first and second priming elements 872a, 872b can include respective arms or sealing segments 873 (individually identified as a first arm 873a of the first priming element 872a and a second arm 873b of the second priming element 872b in FIGS. 8A and 8B) sized and/or otherwise configured to have an interference fit with the outflow channel 764. Additionally, or alternatively, one or both of the arms 873 can include a sealing element (e.g., a seal-forming coating, an O-ring, etc.) and/or another suitable seal-forming configuration. When positioned within the outflow channel 764, the substantially fluid-impermeable seal between priming elements 872 and the outflow channel 764 can be configured to store a vacuum generated within at least a portion of the shunt 136, as described previously with reference to FIG. 6. Movement of one or both of the first and second priming elements 872a, 872b relative to one another and/or the shunt 136, such as at least partially removing one of the arms 873 from the outflow channel 764, can disrupt or breach the substantially fluid-impermeable seal formed between the first and second priming elements 872a, 872b and the outflow channel 764. One or both of the arms 873 can abut the interface portion 762 when positioned within the outflow channel 764. In some embodiments, one or both of the priming elements 872 can include a registration feature 874 (individually identified as a first registration feature 874a of the first priming element 872a and a second registration feature 874b of the second priming element 872b in FIG. 8A) configured to position/orient the priming elements 872a, 872b relative to one another. In the illustrated embodiment, the first registration feature 874a includes a slot or recess and the second registration feature 874b includes tab or projection configured to be positioned within the slot/recess. In other embodiments, the first registration feature 874a can include the tab or projection and the second registration feature 874b can include the slot or recess, and/or one or both of the first and second registration features 874a-b can have another suitable configuration.

To prime the shunt 136, one of the priming elements 872 (e.g., the first priming element 872a) is withdrawn at least partially from the outflow channel 764, as shown in FIGS. 8B and 8D, while the other priming element (e.g., the second priming element 872b) provides a counterforce against the outflow channel interface 762 such that a relative position of the shunt 136 (e.g., with respect to the system 100, 400) is maintained. This movement of one of the priming elements 872 can cause fluid (e.g., priming fluid) to be drawn into the shunt 136, e.g., via the first portion 336a. For example, because the priming elements 872 can form a substantially fluid-impermeable seal with the shunt 136 when positioned within the outflow channel 764, withdrawing one of the priming elements 872 can generate a pressure (e.g., a vacuum) within at least a portion of the shunt 136 (e.g., the outflow channel 764) and cause the generated pressure to be applied to another portion of the shunt 136 (e.g., the flow control assembly 760) to thereby aspirate or otherwise draw fluid (e.g., priming fluid, aqueous, etc.) into the shunt 136. When both of the priming elements 872a, 872b are positioned within the outflow channel 764, the resistance to fluid flow into the shunt is expected to inhibit or prevent fluid low into the shunt. Removing one or both of the priming elements 872 can allow fluid (e.g., priming fluid, aqueous, etc.) to enter at least a portion of the shunt 136 to reduce resistance to initiating fluid flow through the shunt 136, as described previously herein (e.g., with reference to FIG. 6). The remaining priming element (e.g., the second priming element 872b) can be readily removable with minimal force and/or alteration of shunt's placement. This is described in greater detail below with reference to FIGS. 8C and 8D. FIG. 8C, for example, is a cross-sectional view of the shunt 136 and the priming assembly 870 taken along line 8C-8C in FIG. 8A. Referring to FIG. 8C, for example, the tab registration feature 874b of the second priming element 872b is positioned within the slot registration feature 874a of the first priming element 872a, such that the first priming element 872a is positioned between (i) the lateral sides 876a, 876b and upper surface 876c of the second priming element 872b and (ii) the lateral inner sides 878a, 878b and upper inner surface 878c of the outflow channel 764, respectively.

FIG. 8D is a cross-sectional view of the shunt 136 and the priming assembly 870 taken along line 8D-8D in FIG. 8B. Referring to FIG. 8D, when the first priming element 872a is withdrawn from the outflow channel 764, the second priming element 872b can be spaced apart the lateral inner sides 878a, 878b and the upper inner surface 878c of the outflow channel 764 and easier to remove than the first priming element 872a, e.g., by virtue of the tab registration feature 874b being smaller than the recess registration feature 874a.

FIG. 9 is a perspective view of another priming assembly 970 configured in accordance with embodiments of the present technology. At least some aspects of the priming assembly 970 can be generally similar or identical in structure and/or function to the priming assembly 870 of FIGS. 8A-8D. Accordingly, like name and/or reference numbers (e.g., first and second priming elements 972a, 972b versus the first and second priming elements 872a, 872b of FIGS. 8A-8D) are used to indicate generally similar or identical aspects. The priming assembly 970 differs from the priming assembly 870 in that one or both of the first and second priming elements 972a, 972b can include multiple registration features 974. In the illustrated embodiment, for example, the first priming element 972a includes two recessed priming features 974a and the second priming element 972b includes two corresponding protrusion priming features 974b. In other embodiments, one or both of the first and second priming elements 972a, 972b can include at least three, four, five, or another suitable number of registration features.

### EXAMPLES

Several aspects of the present technology are set forth in the following examples:

1. A method for delivering an adjustable shunt to a patient via an implant delivery system, the method comprising:
causing a priming fluid to enter at least a portion of the adjustable shunt;
extending the adjustable shunt from within the implant delivery system, wherein extending the adjustable shunt includes actuating a drive assembly of the implant delivery system to move a driven assembly of the implant delivery system along a longitudinal axis of the implant delivery system, and wherein the driven assembly is configured to carry the adjustable shunt; and
after extending the adjustable shunt from the implant delivery system, positioning the adjustable shunt at a target location within the patient.
   2. The method of example 1 wherein causing the priming fluid to enter at least the portion of the adjustable shunt includes injecting the priming fluid via a priming inlet fluidly coupled to the adjustable shunt.
   3. The method of example 2 wherein injecting the priming fluid includes injecting the priming fluid using a fluid delivery tool and/or a syringe.
   4. The method of example 2 or example 3 wherein injecting the priming fluid via the priming inlet includes injecting the priming fluid via a priming inlet of a cap of the implant delivery system.
   5. The method of any of examples 1-4 wherein causing the priming fluid to enter at least a portion of the adjustable shunt includes creating a pressure gradient between an interior of the adjustable shunt and an exterior of the adjustable shunt.
   6. The method of example 5 wherein creating the pressure gradient includes reducing a pressure within the interior of the adjustable shunt.
   7. The method of any of examples 1-6 wherein causing the priming fluid to enter at least a portion of the adjustable shunt includes causing the priming fluid to enter at least a portion of the adjustable shunt via a vacuum generated within the implant delivery system.
   8. The method of any of examples 1-7, further comprising generating a vacuum within a chamber of the implant delivery system, wherein the adjustable shunt is positioned within the chamber, and wherein causing the priming fluid to enter at least a portion of the adjustable shunt includes drawing the priming fluid into the chamber via the vacuum generated therein.
   9. The method of any of examples 1-8 wherein causing the priming fluid to enter at least the portion of the adjustable shunt includes moving a priming element of a priming assembly positioned at least partially within the adjustable shunt relative to the adjustable shunt.
   10. The method of example 9 wherein moving the priming element includes reducing a pressure within an interior of the adjustable shunt to draw the priming fluid into the adjustable shunt.
   11. The method of example 9 or example 10 wherein moving the priming element includes moving a first priming element of the priming assembly relative to a second priming element of the priming assembly.
   12. The method of any of examples 9-11 wherein moving the priming element includes removing the priming element from an outflow channel of the adjustable shunt.
   13. The method of any of examples 1-12 wherein positioning the adjustable shunt at the target location includes positioning at least a portion of the adjustable shunt posterior to an iris of an eye of the patient.
   14. The method of any of examples 1-12 wherein positioning the adjustable shunt at the target location includes positioning at least a portion of the adjustable shunt anterior to an iris of an eye of the patient.
   15. The method of any of examples 1-14 wherein positioning the adjustable shunt at the target location includes positioning a first portion of the adjustable shunt in a first body region of the patient and positioning a second portion of the adjustable shunt in a second body region of the patient.
   16. The method of example 15, further comprising causing fluid to drain from the first body region toward the second body region.
   17. A priming assembly for an adjustable shunting system, the priming assembly comprising:
a first priming element; and
a second priming element,
wherein the first and second priming elements are configured to be (a) positioned within an outflow channel of the adjustable shunting system and (b) moved relative to one another to cause fluid to enter the adjustable shunting system.
   18. The priming assembly of example 17 wherein the first and second priming elements are movable relative to one another to cause a priming fluid to enter the adjustable shunting system.
   19. The priming assembly of example 17 or example 18 wherein the first and second priming elements are movable relative to one another to cause aqueous to enter the adjustable shunting system.
   20. The priming assembly of example 17 wherein the first priming element includes a first registration feature, and wherein the second priming element includes a second registration feature configured to be positioned at least partially within the first registration feature.
   21. The priming assembly of any of examples 17-20 wherein the first and second priming elements are configured to form a substantially fluid-impermeable seal with the outflow channel.
   22. The priming assembly of example 21 wherein movement of one or both of the first and second priming elements relative to one another disrupts the substantially fluid-impermeable seal.
   23. The priming assembly of example 21 or example 22 wherein movement of one or both of the first and second priming elements relative to one another changes a pressure within an interior of the adjustable shunting system to draw the fluid into the adjustable shunting system.
   24. The priming assembly of any of examples 17-23 wherein the first and second priming elements are configured to contact an interface portion of a flow control assembly of the adjustable shunting system when positioned within the outflow channel.
   25. A delivery system for use with an adjustable shunt for treating a patient, the delivery system comprising:
a housing including a tip, wherein the tip defines a distal terminus of the delivery system;
a drive assembly movably coupled to the housing; and
a driven assembly operably coupled to the drive assembly and configured to (i) be releasably coupled to the adjustable shunt, and (ii) move relative to the housing and cause a corresponding movement of the driven assembly to transition the delivery system between a first state and a second state,
wherein
   in the first state, the driven assembly is configured such that the adjustable shunt is completely within the housing, and
   in the second state, the driven assembly is configured such that at least a portion of the adjustable shunt extends distally beyond the distal terminus of the housing.
      26. The delivery system of example 25 wherein:
the housing extends along a longitudinal axis;
the drive assembly is rotatably coupled to the housing;
the driven assembly is slidably coupled to the housing; and
the driven assembly is configured to move along the longitudinal axis of the housing in response to rotational movement of the drive assembly.
   27. The delivery system of example 25 or example 26 wherein the drive assembly includes a threaded drive shaft threadably coupled to the housing.
   28. The delivery system of any of examples 25-27 wherein the drive assembly includes an annular protrusion, and wherein the driven assembly includes an annular slot configured to receive the annular protrusion.
   29. The delivery system of example 28 wherein the annular protrusion is configured to rotate within the annular slot such that the driven assembly does not rotate in response to rotational movement of the drive assembly.
   30. The delivery system of any of examples 25-29 further comprising a shunt coupling member configured to releasably couple the adjustable shunt to the driven assembly.
   31. The delivery system of example 30 wherein the shunt coupling member is configured to be positioned at least partially within the adjustable shunt.
   32. The delivery system of example 31 wherein the shunt coupling member is configured to facilitate priming of the adjustable shunt with priming fluid before delivery of the adjustable shunt within the patient.
   33. The delivery system of example 32 wherein the shunt coupling member is configured to cause the priming fluid to enter at least a portion of the adjustable shunt when the shunt coupling member is moved relative to the adjustable shunt.
   34. The delivery system of example 32 or example 33 wherein the shunt coupling member includes a priming assembly, wherein the priming assembly includes a priming element configured to be positioned within an outflow channel of the adjustable shunt.
   35. The delivery system of example 34 wherein the priming element is a first priming element, the priming assembly further comprising a second priming element, wherein the first and second priming elements are movable relative to one another and the adjustable shunt to cause the adjustable shunt to be primed with the priming fluid.
   36. The delivery system of any of examples 25-35, further comprising a priming port configured to fluidly couple the adjustable shunt to a priming fluid source when the adjustable shunt is carried by the delivery system.
   37. The delivery system of example 36, further comprising a cap configured to be releasably coupled to the housing at least partially around the tip, wherein the cap includes the priming port.
   38. The delivery system of example 37 wherein the cap is configured to contain a vacuum generated therein and receive priming fluid drawn from the priming fluid source through the priming port via the vacuum.
   39. The delivery system of any of examples 36-38 wherein the tip includes (i) an opening defining the distal terminus and (ii) the priming port, wherein the priming port is opposite the opening.
   40. The delivery system of any of examples 25-39 wherein the adjustable shunt is an adjustable intraocular shunt configured to be positioned in an eye of a patient.

### CONCLUSION

The above detailed description of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, any of the features of the adjustable shunts described herein may be combined with any of the features of the other adjustable shunts described herein and vice versa. Moreover, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions associated with intraocular shunts have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/we claim:

1. A method for delivering an adjustable shunt to a patient via an implant delivery system, the method comprising:
causing a priming fluid to enter at least a portion of the adjustable shunt;
extending the adjustable shunt from within the implant delivery system, wherein extending the adjustable shunt includes actuating a drive assembly of the implant delivery system to move a driven assembly of the implant delivery system along a longitudinal axis of the implant delivery system, and wherein the driven assembly is configured to carry the adjustable shunt; and
after extending the adjustable shunt from the implant delivery system, positioning the adjustable shunt at a target location within the patient.

2. The method of claim 1 wherein causing the priming fluid to enter at least the portion of the adjustable shunt includes injecting the priming fluid via a priming inlet fluidly coupled to the adjustable shunt.

3. The method of claim 2 wherein injecting the priming fluid includes injecting the priming fluid using a fluid delivery tool and/or a syringe.

4. The method of claim 2 wherein injecting the priming fluid via the priming inlet includes injecting the priming fluid via a priming inlet of a cap of the implant delivery system.

5. The method of claim 1 wherein causing the priming fluid to enter at least a portion of the adjustable shunt includes creating a pressure gradient between an interior of the adjustable shunt and an exterior of the adjustable shunt.

6. The method of claim 5 wherein creating the pressure gradient includes reducing a pressure within the interior of the adjustable shunt.

7. The method of claim 1 wherein causing the priming fluid to enter at least a portion of the adjustable shunt includes causing the priming fluid to enter at least a portion of the adjustable shunt via a vacuum generated within the implant delivery system.

8. The method of claim 1, further comprising generating a vacuum within a chamber of the implant delivery system, wherein the adjustable shunt is positioned within the chamber, and wherein causing the priming fluid to enter at least a portion of the adjustable shunt includes drawing the priming fluid into the chamber via the vacuum generated therein.

9. The method of claim 1 wherein causing the priming fluid to enter at least the portion of the adjustable shunt includes moving a priming element of a priming assembly positioned at least partially within the adjustable shunt relative to the adjustable shunt.

10. The method of claim 9 wherein moving the priming element includes reducing a pressure within an interior of the adjustable shunt to draw the priming fluid into the adjustable shunt.

11. The method of claim 9 wherein moving the priming element includes moving a first priming element of the priming assembly relative to a second priming element of the priming assembly.

12. The method of claim 9 wherein moving the priming element includes removing the priming element from an outflow channel of the adjustable shunt.

13. The method of claim 1 wherein positioning the adjustable shunt at the target location includes positioning at least a portion of the adjustable shunt posterior to an iris of an eye of the patient.

14. The method of claim 1 wherein positioning the adjustable shunt at the target location includes positioning at least a portion of the adjustable shunt anterior to an iris of an eye of the patient.

15. The method of claim 1 wherein positioning the adjustable shunt at the target location includes positioning a first portion of the adjustable shunt in a first body region of the patient and positioning a second portion of the adjustable shunt in a second body region of the patient.

16. The method of claim 15, further comprising causing fluid to drain from the first body region toward the second body region.

17. A priming assembly for an adjustable shunting system, the priming assembly comprising:
a first priming element; and
a second priming element,
wherein the first and second priming elements are configured to be (a) positioned within an outflow channel of the adjustable shunting system and (b) moved relative to one another to cause fluid to enter the adjustable shunting system.

18. The priming assembly of claim 17 wherein the first and second priming elements are movable relative to one another to cause a priming fluid to enter the adjustable shunting system.

19. The priming assembly of claim 17 wherein the first and second priming elements are movable relative to one another to cause aqueous to enter the adjustable shunting system.

20. The priming assembly of claim 17 wherein the first priming element includes a first registration feature, and wherein the second priming element includes a second registration feature configured to be positioned at least partially within the first registration feature.

21. The priming assembly of claim 17 wherein the first and second priming elements are configured to form a substantially fluid-impermeable seal with the outflow channel.

22. The priming assembly of claim 21 wherein movement of one or both of the first and second priming elements relative to one another disrupts the substantially fluid-impermeable seal.

23. The priming assembly of claim 21 wherein movement of one or both of the first and second priming elements relative to one another changes a pressure within an interior of the adjustable shunting system to draw the fluid into the adjustable shunting system.

24. The priming assembly of claim 17 wherein the first and second priming elements are configured to contact an interface portion of a flow control assembly of the adjustable shunting system when positioned within the outflow channel.

25. A delivery system for use with an adjustable shunt for treating a patient, the delivery system comprising:
a housing including a tip, wherein the tip defines a distal terminus of the delivery system;
a drive assembly movably coupled to the housing; and
a driven assembly operably coupled to the drive assembly and configured to (i) be releasably coupled to the adjustable shunt, and (ii) move relative to the housing and cause a corresponding movement of the driven assembly to transition the delivery system between a first state and a second state,
wherein
in the first state, the driven assembly is configured such that the adjustable shunt is completely within the housing, and
in the second state, the driven assembly is configured such that at least a portion of the adjustable shunt extends distally beyond the distal terminus of the housing.

26. The delivery system of claim 25 wherein:
the housing extends along a longitudinal axis;
the drive assembly is rotatably coupled to the housing;
the driven assembly is slidably coupled to the housing; and
the driven assembly is configured to move along the longitudinal axis of the housing in response to rotational movement of the drive assembly.

27. The delivery system of claim 25 wherein the drive assembly includes a threaded drive shaft threadably coupled to the housing.

28. The delivery system of claim 25 wherein the drive assembly includes an annular protrusion, and wherein the driven assembly includes an annular slot configured to receive the annular protrusion.

29. The delivery system of claim 28 wherein the annular protrusion is configured to rotate within the annular slot such that the driven assembly does not rotate in response to rotational movement of the drive assembly.

30. The delivery system of claim 25 further comprising a shunt coupling member configured to releasably couple the adjustable shunt to the driven assembly.

31. The delivery system of claim 30 wherein the shunt coupling member is configured to be positioned at least partially within the adjustable shunt.

32. The delivery system of claim 31 wherein the shunt coupling member is configured to facilitate priming of the adjustable shunt with priming fluid before delivery of the adjustable shunt within the patient.

33. The delivery system of claim 32 wherein the shunt coupling member is configured to cause the priming fluid to enter at least a portion of the adjustable shunt when the shunt coupling member is moved relative to the adjustable shunt.

34. The delivery system of claim 32 wherein the shunt coupling member includes a priming assembly, wherein the priming assembly includes a priming element configured to be positioned within an outflow channel of the adjustable shunt.

35. The delivery system of claim 34 wherein the priming element is a first priming element, the priming assembly further comprising a second priming element, wherein the first and second priming elements are movable relative to one another and the adjustable shunt to cause the adjustable shunt to be primed with the priming fluid.

36. The delivery system of claim 25, further comprising a priming port configured to fluidly couple the adjustable shunt to a priming fluid source when the adjustable shunt is carried by the delivery system.

37. The delivery system of claim 36, further comprising a cap configured to be releasably coupled to the housing at least partially around the tip, wherein the cap includes the priming port.

38. The delivery system of claim 37 wherein the cap is configured to contain a vacuum generated therein and receive priming fluid drawn from the priming fluid source through the priming port via the vacuum.

39. The delivery system of claim 36 wherein the tip includes (i) an opening defining the distal terminus and (ii) the priming port, wherein the priming port is opposite the opening.

40. The delivery system of claim 25 wherein the adjustable shunt is an adjustable intraocular shunt configured to be positioned in an eye of a patient.
